(19) United States
(12) Patent Application Publication
(10) Pub. No.: US 2020/0246500 A1
(43) Pub. Date: Aug. 6, 2020

(54) MOISTURE DETECTOR USING WATER-SOLUBLE CONDUCTOR

(71) Applicants: Gerald Rogers, Dallas, TX (US); Fahim Shaikh, Garland, TX (US)

(72) Inventors: Gerald Rogers, Dallas, TX (US); Fahim Shaikh, Garland, TX (US)

(21) Appl. No.: 16/268,165

(22) Filed: Feb. 5, 2019

(51) Int. Cl. A61L 15/56; A61F 13/42; A61F 13/49; A61L 15/22; A61F 13/15

(52) U.S. Cl. CPC A61L 15/56; A61F 13/42; A61F 13/49; A61F 2013/15829; A61F 13/15764; A61F 2013/424; A61F 2013/426; A61L 15/225

(57) ABSTRACT

A disposable moisture detection component manufactured from a water soluble conductive polymer film-forming and printable mixture with a resistivity of 100 ohms or less. Moisture detection component is embedded in incontinent articles such as diapers and bedpans. Conductive polymer dissolves in the presences of urine or feces and when connected to an electronic circuit, provides diaper status to a caregiver.

### FIELD OF THE INVENTION

The invention relates to a water-soluble conductor embedded in personal hygiene absorbent articles of the type worn in the crotch region of a wearer to absorb body excreta (diapers). The water-soluble conductor is used to detect soiled incontinent hygiene articles with changing conductor resistance due to conductor dissolving and dispersing while in contact with excreta.

### BACKGROUND OF THE INVENTION

Diapers are typically fabricated with an absorbent core between a topsheet on the wearer-facing side and the liquid impermeable backsheet on the garment-facing side of the diaper. As the absorbent core becomes saturated with urine, it tends to sag down in the crotch region of the wearer due to the weight of the fluid. This may cause loss of contact of the article along the thighs of the wearer and increase the possibility of leakages. While elastic waist bands and other elasticized parts such as barrier leg cuffs are commonly used to maintain contact and fit, these solutions are limited and leakage can still occur, especially if the diaper was not put in place correctly or was displaced out of position by the wearer.

Caregivers monitoring diapered individuals use sight, smell and touch to determine if the diaper is wet without any indication of the degree of wetness or how long the individual has been wet. A visual wetness indicator fabricated by printing or otherwise fabricating a chemical stripe on the center of the backsheet along the length of the diaper, changes color when in contact with urine. Even though the stripe is the length of the diaper, only the portion in contact with the excreta changes color which my not be visible to the caregiver.

There are electronic diaper wetness monitors with a moisture sensor on the back sheet such as parallel carbon stripes where the resistance between the stripes decreases as a function of the length of the stripes in contact with the urine. The excreta must penetrate the absorbent core and provide a bridge between the carbon stripes to be recognized or sensed as a wet diaper.

These backsheet sensors signal a wet diaper when the absorbent core is saturated and excreta is covering a significant portion of the sensor. And in particular, sensors relying on a change in resistance have an additional sensing problem with the urine varying in conductivity by as much as 5X. The wide variance in conductivity, and thus resistance, requires a greater length of the carbon stripes to be covered with urine in order to indicate the diaper is wet. There is a high probability the excreta will escape the diaper and soil bedding and/or cloths well before the system is signaling a wet diaper.

Another type electronic sensor uses Frequency Identification (RFID) tags placed between the topsheet and wearer to detect a wet diaper such as found in U.S. Pat. No. 10,069,205. The RFID tag antenna changes resonant frequency when setting on wet substrate as compared to a dry substrate. The RFID reader identifies the change in frequency signaling a wet diaper.

The RFID sensor on the topsheet is sensitive to stress incontinence due to weak pelvic and sphincter muscles allowing leakage during laughing, sneezing, coughing as a few examples of stress incontinence signaling a false wet diaper to the caregiver causing a premature diaper change. RFID requires a hand held reader that needs to be within two feet or less from the RFID tag in order to read the signal (active the tag for transmitting status), not a significant advantage compared to the chemical sensor changing color.

The Applicants' invention is a water soluble conductor when connected to an electronic monitoring circuit to detect the changes in the water soluble conductor minimizes the probability of leakage as well as unexpectedly sending a false signal prior to full utilization of the diaper.

### SUMMARY OF INVENTION

A first aspect, the invention has a Water Soluble Conductor (WSC) deposited on a non-woven fabric for incorporating in the manufacturing of wetness sensored diapers. WSC means any flexible material composition of conducting elements or components like silver, gold, carbon black, carbon nanotubes, or graphene that conducts electric current or any combination thereof, mixed with a water soluble component such as polyvinyl alcohol copolymers, polyethyloxyazoline, polyvinyl pyrrolidone, polyvinyl alcohol, polyacrylamide, polyglycols or polyacrylic acid or any mixtures thereof. The WSC in instances includes a thickness agent, surface active agent and/or fast dry agent for a flexible, conductor that disperses when wet creating open or very high resistance circuit.

An electronic monitor attached to the water-soluble conductor (WSC) to monitor the WSC for a change in resistance determining the fabricated sensored diaper has been soiled. Monitor means any electronic circuit with power to pass current through the WSC and monitor changes in conductivity of the WSC and signal a caregiver visually illuminating or flashing a LED for a wet or soiled incontinent garment or, use a device capable of signaling a caregiver with a RF data transmission containing the status of the incontinent garment, wet or dry.

### BRIEF DESCRIPTION OF THE DRAWINGS

FIG. 1 is a two dimensional view of a fabricated sensor.

FIG. 2 shows a converter-bonding machine attaching a sensor to a fabric substrate

FIG. 3 depicts an off set fabric printer

FIG. 4 is a side view of fabric printing machine with input and output rolls

FIG. 5 is a top view section of a sensor attached to a liquid permeable fabric

FIG. 6 is an exploded perspective view of several layers of a disposable diaper

FIG. 7 shows a sensored diaper product

### DETAIL DESCRIPTION OF THE INVENTION

The invention is a solution for managing incontinence with electronic wetness monitor to provide caregivers notice of a soiled incontinent garment or pad and avoid leakage resulting in soiled clothing and/or furniture items such as chairs and beds. Invention enables caregivers to quickly remove the soiled diaper minimizing the potential for rash and eventual urinary track infection associate with prolonged periods of time in a soiled diaper.

The invention is a novel way to manufacture a moisture sensor combining water-soluble polymeric films with conductive fine particles where the water-soluble film disintegrates in water (urine and/or feces) dispersing the conductive particles. Urine and/or feces disintegrates the conductive polymeric film disrupting or eliminating conductivity in the portion of the film that lays bare to urine and/or feces and the disruption is detected by an electronic sensor connected to the film. Disruption means an increase in resistivity. The water-soluble conductive polymer film-forming and printable composition mixes polyvinyl pyrrolidone, polyvinyl alcohol and aliphatic polyethers or polyethylene glycols or a combination thereof with conductive gold, silver, carbon black, graphene, or graphite or a combination thereof manufacturing a water soluble conductive material for fabricating moisture sensors.

Flexibility of the polymeric film is improved with a plasticizer 5% wt to 15% wt such as water-soluble polyglycols. Additionally, thickening agent such as sodium carboxymethylcellulose can be used to provide a viscosity of 2 poise to about 1500 poise for casting or printing the conductive polymer.

One embodiment depicted in FIG. 1 is a cutout WSC sensor 100 from a continuous roll of sensors 5000 meters or longer. Sensor 100 is fabricated with water soluble material such as polyvinyl alcohol (PVOH) with carbon black mixture fabricated in a thin film about 80 microns thick from a viscous WSC material printed or other wise deposited on a substrate. Two conductors 101 and 102 are parallel and separated by space S1 104. Conductors 101 and 102 are 1 mm to 50 mm wide and in particular 5 mm to 10 mm spaced S1 104 from 5 MM to 100 mm and in particular 10 to 25 mm. Link 103 and 103a separated by length L1 106 where L1 106 is a function of the various disposable diaper sizes. Link 103 width W2 107 is at a minimum the width of the stripes 101 and 102, and designed to provide continuity between the conductors 101 and 102.

WSC is designed for a resistively per square of 20 ohms to 100 ohms and preferably 25 to 50 ohms per square at a thickness of 40 microns to 80 microns. WSC must be flexible to accommodate the wearer's movement in and with the incontinent garment. Plasticizers are added to the dimethylformamide, dimethylacetamide, ethylene glycol, propylene glycol and water-soluble polyglycols. Plasticizers improve the flexibility and resistance to cracking or flexing of the mixed polymer films of this invention. Flexible means twisting and folding without changing the resistance per square of the dry WSC sensor 100.

One fabrication of sensor 100, referred to as stamped water soluble conductor (SWSC), uses POVH carbon black film with width W1 105 stamped or otherwise cutout with spacing S1 104 and length L1 106 centered on W1 105 repeating every (L1 106+W2 107) length for 5000 meters or more.

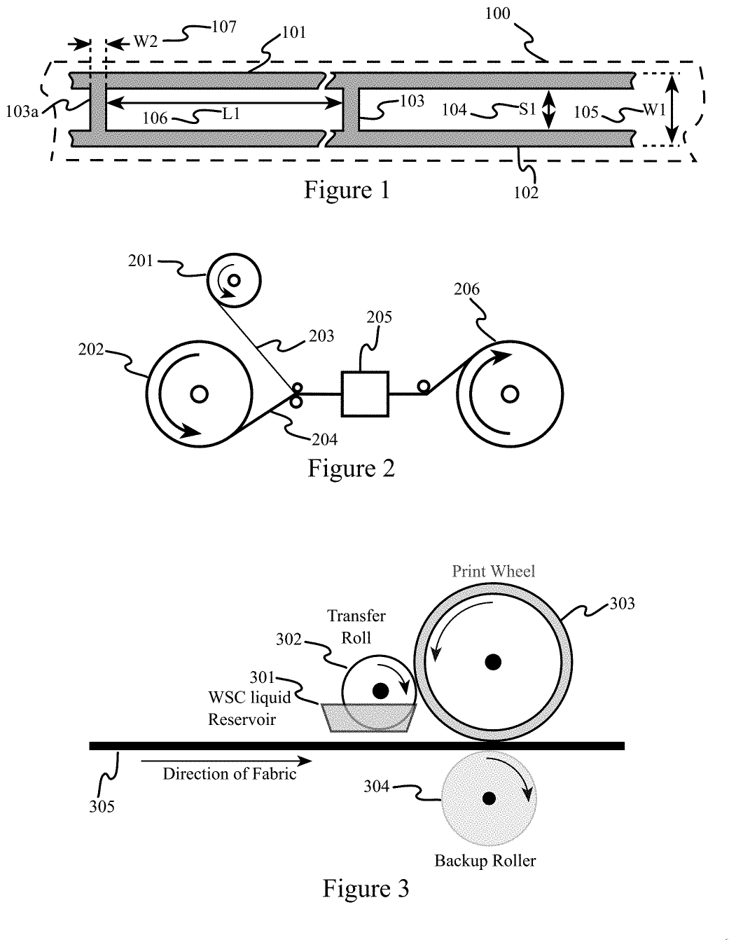

FIG. 2 depicts a conversion machine for combining the SWSC film 203 from a roll 201 with liquid permeable fabric 204 from a roll 202, and bonded together in bonder 205. The sensor bonded to the liquid permeable fabric and shown in FIG. 5 as a cutout of a partial sensored fabric 500, is finished and spooled on roll 206 for use by manufacturer of a disposable diaper or disposable incontinent underpads for beds and furniture.

A preferred embodiment of the invention is a liquid WSC that can be printed on liquid permeable fabric 502 using a low cost printing machine as depicted in FIG. 3. A viscous WSC liquid from about 100 cps to about 10,000 cps, including from about 100 cps to about 1000 cps is transported to the WSC reservoir 301. Print wheel 303 has a sensor 100 pattern offset from the surface of the print wheel 303. WSC liquid is applied with transfer roller 302 to the offset print wheel 303. Print wheel 303 with WSC liquid on the surface of offset sensor 100 rolls on to fabric 305 with backup roller holding the fabric against the print wheel 303 where the WSC liquid is transferred to the fabric 305 at a WSC thickness of 40 to 200 microns.

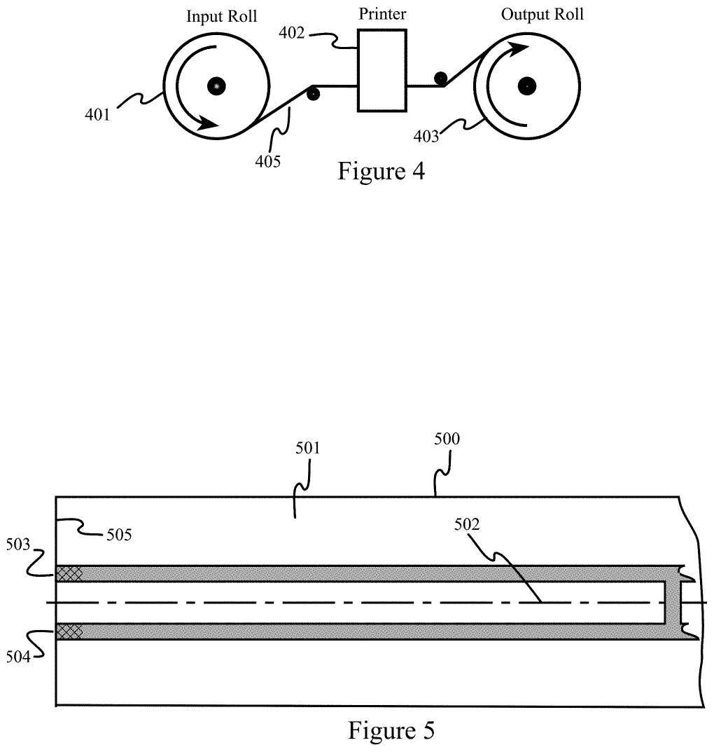

FIG. 4 shows a low cost converter with a printer 402 that could be an ink jet printer, spray printer or offset printer such as FIG. 4, between the input roll 401 and output roll 403 applying WSC liquid on water permeable fabric 404.

FIG. 5 shows a top view cutout of a complete sensor 100 fabricated on a water permeable fabric 501 cut from a roll of sensors at least 5000 meters long. Sensor 100 is attached to fabric 501 as a SWSC film bonded, as described with FIG. 2, or otherwise attached to the fabric or SWC printed on the fabric, as described with FIG. 3 and FIG. 4. Sensor 100 is centered 502 on fabric 501 along longitudinal length of fabric 501. Ends of the WSC stripes 101a and 102a are shown in FIG. 5 extending to the edge of the diaper 503 and 504 for electrical connection to an electronic monitor.

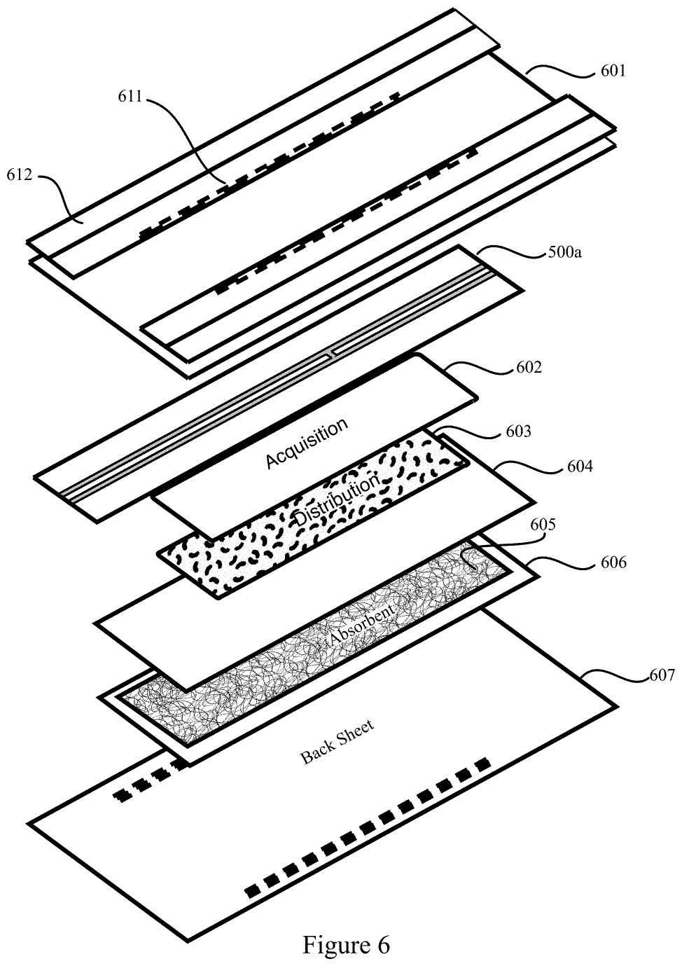

FIG. 6 is a perspective view of some of the layers used in the manufacture of a typical disposable diaper. The wearer-facing side of the diaper comprises a liquid permeable topsheet 601, the garment facing surface comprises a liquid impermeable backsheet 607, and an absorbent core made up of absorbent layer 605 sandwiched between covers 604 and 606. The perspective view comprises two liquid management layers; a liquid acquisition layer 602; and liquid distribution layer 603. Liquid management layers wick liquid away from the top sheet 601 keeping the wearer dry (free from liquid) directing the liquid 603 to the absorbent core 604, 605 and 606. Liquid acquisition layer 602, liquid distribution layer 603, and layer 604 are combined in some diapers and referred to as a surge layer. Sensor fabric 500a is added to the diaper construction before the liquid management material layer 602 and after topsheet 601. The sensor is the length of the top sheet 601 and is at least as wide as the core 604. In particular, the sensor 100 could be attached or printed to the backside of topsheet 601 running the length with the link 103 positioned in the rear just above the anal area.

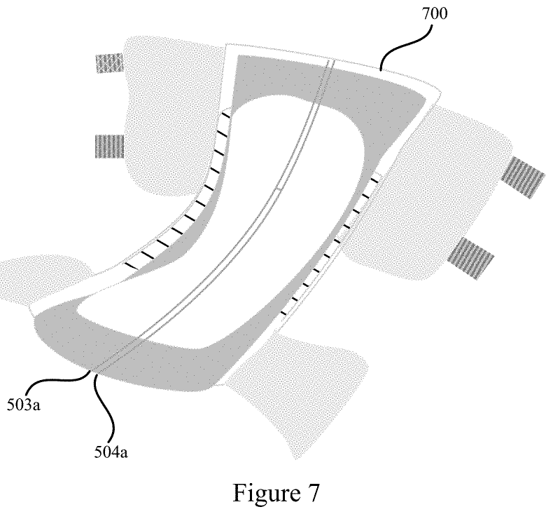

FIG. 7 illustrates a finished diaper incorporating the layers identified in FIG. 6 including layer 500a with a WSC available for contact at 503a and 504a. Connection to the WSCs at 503a and 504a is made with a connector clamp capable of penetrating the top and/or back sheet layers to connect to the WSC 503a and 504a. A particular connector clamp has conductive pins sharp enough to penetrate the topsheet and the backsheet making contact with the WSC stripes 101 and 102 at 503a and 504a. The pins are connected to an electronic monitor for detecting resistance between 503a and 504a, with low resistance indicating a dry diaper while high resistance, more than 2× dry resistance indicates a wet diaper, results from the dispersement of the WSC in moisture.

Urine is wicked from the top sheet 601 by the acquisition layer 602 past sensor layer 500a into absorption layer 605. Urine passing the WSC in the sensor 100, erodes the WSC stripes increasing the resistance, but does not disperse the carbon, which would create an open circuit. In one instance, the resistance between WSC contacts 503a and 504a was measured after 50 ml of synthetic urine was poured on the diaper in the crotch area, followed by additional 50 ml volume poured on the diaper while measuring resistance during and after each application of 50 ml of urine. The WSC opened after 1000 ml to 1500 ml of synthetic urine was poured on the crotch area of the diaper 700.

WSC sensor 100 can be used to monitor the time and volume of urine and feces in diapers 700 providing wearer's incontinent garment status to a caregiver. Relationship between excreta volume and WSC sensor 100 resistances is programmed into a monitor to provide the caregiver the time for various episodes of urination or bowel movement. Caregiver will have the information regarding the patient's incontinence to make decisions for the best health of the patient. Wetness not sufficient to trigger an alarm under normal circumstances would be detrimental to the patient if the wetness were prolonged in time. The WSC sensor 100 along with an attached monitor would allow or flag the caregiver to replace the soiled incontinent garment with a clean dry garment.

1. An water soluble conductive polymer film-forming and printable mixture comprising:
a water-soluble polymer about 5 wt % to about 40 wt % by weight selected from the group consisting of polyethyloxyazoline, polyvinyl pyrrolidone, polyvinyl alcohol, polyacrylamide, polyglycol, and polyacrylic acid or combination thereof;
electrically conducting particles in the amount of 5 wt % to 60 wt % selected from the group consisting of conductive metal particles, carbon black particles, graphite particles, graphene particles;
an effective solvating amount of water to dissolve water-soluble polymer;
each of said amounts being based upon the total weight of said mixture resulting in a water-soluble conductor with 20 ohms per square to 100 ohms per square resistivity.

2. The mixture of claim 1 containing from 5% wt to 15% wt parts by weight plasticizer selected from the group consisting of dimethylformamide, dimethylacetamide, ethylene glycol, propylene glycol, glycerol and water-soluble polyglycols. Preferred plasticizers are dimethylformamide, dimethylacetamide, ethylene glycol, propylene glycol and water-soluble polyglycols.

3. The mixture of claim 1 containing up to 5% with 1% wt to 3% wt preferred water soluble or water dispersible polymeric thickening agents and mixtures.

4. The mixture of claim 1 thickness from about 20 microns to 150 microns and preferably 30 microns to 90 microns deposited or attached on a fabric substrate.

5. The mixture of claim where electrical conducting particles are less than 1000 nanometers with a concentration of said conducting particles less than 100 ohms per square in a film 80 microns thick.

6. Water soluble conducting film comprising:
a water-soluble polymer mixed with electrical conducting particles less than 1000 nanometers with a concentration of said conducting particles less than 100 ohms per square in a film 80 microns thick;
having moisture sensors die cut from the film producing parallel stripes 3 to 10 millimeters wide, spaced 5 to 25 millimeters apart with the stripes connected with a link 103 conductor;
wherein moisture sensor is centered the length of the incontinent garment with the electronic link connector located above the anus of a wearer of the incontinent garment;
wherein manufactured incontinent garment incorporates the moisture sensor with the stripes available for electronic contact at the front top edge; and
a portion of the moisture sensor stripes at the edge of the garment are accessible to determine change in sensor resistance when penetrated by moisture whereby the water-soluble polymer stripe or stripes dissolve and the conductive particles disperse resulting in an increase of sensor resistance of at least 2× when compared to a dry sensor.

7. A moisture sensor embedded in a diaper to measure the wetness of the diaper comprising:
conductive particles suspended in a water-soluble film in a concentration producing 100 ohms per square or less;
water-soluble film dissolves in contact with urine reducing the cross section area of the film increasing the resistance;
water-soluble film erosion from urine is calibrated for particular urine volumes wicked past the film and the associated resulting resistance;
a table of values of volume and resulting resistance is used in the electronic monitoring device to record and display or communicate degree of diaper wetness.
